(19) United States
(12) Patent Application Publication
(10) Pub. No.: US 2024/0210413 A1
(43) Pub. Date: Jun. 27, 2024

(54) METHOD AND KIT FOR DETECTING CONTENT OF WHEAT GLUTEN IN FOOD

(71) Applicant: Zhejiang Gongshang University, Hangzhou (CN)

(72) Inventors: Linglin FU, Hangzhou (CN); Jinru ZHOU, Hangzhou (CN); Yanzhuo DU, Hangzhou (CN); Jiangbing SHUAI, Hangzhou (CN); Xiaofeng ZHANG, Hangzhou (CN); Ke LI, Hangzhou (CN)

(73) Assignee: Zhejiang Gongshang University, Hangzhou (CN)

(21) Appl. No.: 18/460,739

(22) Filed: Sep. 5, 2023

(30) Foreign Application Priority Data
Dec. 23, 2022 (CN) 202211666745.2

(51) Int. Cl. G01N 33/68; G01N 21/65; G01N 33/53; G01N 33/543

(52) U.S. Cl. CPC G01N 33/6848; G01N 21/658; G01N 33/5308; G01N 33/54386; G01N 2333/415

(57) ABSTRACT

A method and a kit for detecting a content of wheat gluten in food are provided. The kit includes a Raman enhanced substrate solution and a quartz sheet, where the Raman enhanced substrate solution includes a gold nanorod dimer and a Raman signal molecule, where the gold nanorod dimer is formed by self-assembling a gold nanorod functionalized by aptamer, and a gold nanorod functionalized by an aptamer complementary chain. The kit has simple and convenient operation and a convenient instrument, and can quickly, stably and sensitively detect the content of the wheat gluten in the food.

### CROSS REFERENCE TO THE RELATED APPLICATIONS

This application is based upon and claims priority to Chinese Patent Application No. 202211666745.2, filed on Dec. 23, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBXMCX020_Sequence_Listing.xml, created on Aug. 16, 2023, and is 2,952 bytes in size.

### TECHNICAL FIELD

The present disclosure relates to the technical field of biological analysis, and in particular to a method and kit for detecting a content of wheat gluten in food.

### BACKGROUND

Wheat, the most commonly used raw food material, is widely used in food and food industry. However, serving as one of eight common types of allergic food, the wheat can cause serious allergic reactions. Wheat allergy has become a global food safety problem. Wheat gluten has been identified as a protein that causes chylous diarrhea in the wheat, and mainly includes gliadin and glutenin, which approximately account for 80%-85% of a total protein of the wheat. However, there is no effective method to treat the wheat allergy at present. Accordingly, food containing allergenic components are strictly avoided to prevent the wheat allergy. In order to protect vulnerable populations, governments and international organizations have successively revised and introduced legal acts regulating labeling of allergens in food, and have strictly regulated the labeling of allergen components on food labels. For example, countries such as the United States, the European Union, and Canada require mandatory labeling of food allergen wheat components. As a result, detection of the wheat gluten is closely related to production and labeling of the food. Research on detection of the food allergen components in the food mainly relies on a protein based method (such as enzyme linked immunosorbent assay (ELISA) detection) and a gene based method (such as polymerase chain reaction (PCR) detection), which have the advantages of high detection sensitivity and accuracy, but have limitations such as long time consumption, large and expensive apparatuses, and the need for professional operators. In view of that, it is necessary to establish a quick, portable, accurate and sensitive method for detecting wheat gluten.

### SUMMARY

The present disclosure aims to at least solve one of the technical problems in the related art to some extent. To this end, an objective of the present disclosure is to provide a method and a kit for detecting a content of wheat gluten in food. The kit has simple and convenient operation and a convenient instrument, and may quickly, stably and sensitively detect the content of the wheat gluten in the food.

To this end, in a first aspect of the present disclosure, the present disclosure provides a kit for detecting a content of wheat gluten in food. The kit includes a Raman enhanced substrate solution and a quartz sheet, where the Raman enhanced substrate solution includes a gold nanorod dimer and a Raman signal molecule, where the gold nanorod dimer is formed by self-assembling a gold nanorod functionalized by aptamer, and a gold nanorod functionalized by an aptamer complementary chain.

According to the kit of the present disclosure, the gold nanorod dimer is prepared with a self-assembly technology as a Raman probe. On the basis of specific recognition of wheat gliadin and the aptamer, the gold nanorod dimer is depolymerized to form a single dispersed gold nanorod. In a sensing system, a Raman intensity is proportional to a yield of the gold nanorod dimer. Therefore, a concentration of the wheat gluten is inversely proportional to the Raman signal intensity. The kit has simple and convenient operation and a convenient instrument, and may quickly, stably and sensitively detect the content of the wheat gluten in the food.

Alternatively, the aptamer has a nucleic acid sequence as shown in SEQ ID NO: 1, and the aptamer complementary chain has a nucleic acid sequence as shown in SEQ ID NO: 2.

Alternatively, a molar ratio of the gold nanorod to the aptamer or the aptamer complementary chain is 1:60.

Alternatively, a length-diameter ratio of the gold nanorod is 3.7.

In a second aspect of the present disclosure, the present disclosure provides a method for detecting a content of wheat gluten in food by using the kit. The method includes:
adding 20 μL of Raman enhanced substrate solution into 1 μL of sample solution to be detected, carrying out incubation at a room temperature to form a mixed solution, dropwise adding 10 μL of mixed solution on a quartz sheet, and drying the quartz sheet; and
placing a dried quartz sheet into a Raman spectrometer to measure a Raman signal intensity, and calculating the total content of the wheat gluten in a sample to be detected.

The detection method according to an example of the present disclosure may quickly and sensitively detect the content of the wheat gluten in the food.

Additional aspects and advantages of the present disclosure will be set forth partially in the following description, which will become obvious in the following description, or may be learned by practice of the present disclosure.

### BRIEF DESCRIPTION OF THE DRAWINGS

FIG. 1 is a standard curve of detection of wheat gluten according to an example of the present disclosure; and

FIG. 2 shows specificity of a standard sample detected by a kit according to an example of the present disclosure.

### DETAILED DESCRIPTION OF THE EMBODIMENTS

The technical solutions of the present disclosure will be described below by means of specific particular examples. It should be understood that one or more method steps mentioned in the present disclosure do not exclude the presence of other method steps before and after the combined steps, or the possibility of inserting other method steps between these explicitly mentioned steps; and it should further be understood that these examples are only used to illustrate the present disclosure and are not used to limit the scope of the present disclosure. Moreover, unless otherwise specified, the number of each method step is only a convenient tool for identifying each method step, and is not intended to limit the arrangement order of the method steps or limit the implementable scope of the present disclosure. Changes or adjustments in relative relations of the numbers shall also be considered as the implementable scope of the present disclosure without substantial changes in the technical content.

In order to better understand the above technical solutions, illustrative examples of the present disclosure will be described in more detail. Although the illustrative examples of the present disclosure are shown, it should be understood that the present disclosure can be implemented in various forms and should not be limited by the examples described herein. On the contrary, these examples are provided to provide a more thorough understanding of the present disclosure and to fully convey the scope of the present disclosure to those skilled in the art.

Test materials used in the present disclosure are all ordinary commercially available products that may be purchased in the market.

The present disclosure will be described below with reference to the specific examples. It should be noted that these examples are only descriptive and do not limit the present disclosure in any way.

### EXAMPLE 1

### Preparation of Gold Nanorod Dimer

A gold nanorod dimer was prepared with a self-assembly method, and specific steps were as follows:
(1) 12 μL of ultra pure water, 8 μL of 0.2% sodium dodecyl sulfate (SDS), 4 μL of 10×tetrabromoethane (TBE) buffer solution, 2.4 μL of 1 μM aptamer (Apt) solution (19.9% of tris-edta (TE) buffer was added per optical density (OD) of aptamer to prepare 100 μM of mother solution, and the mother solution was diluted to 1 μM) or 2.4 μL of 1 μM aptamer complementary solution (RC) (22.6 μL of TE buffer was added per OD of aptamer complementary chain to prepare 100 μM of mother solution, and the mother solution was diluted to 1 μM) were added into 20 μL of gold nanorod (2 nM), mixed, oscillated and incubated for 12 h at a room temperature.
(2) A reaction solution in step (1) was centrifuged for 5 min at 5000 rpm, and a supernatant was discarded. A reactant was washed with 0.5×TBE solution containing 0.02% SDS once to remove excess Apt or RC, and a precipitate was dispersed into 10 μL of 0.5×TBE solution containing 0.02% SDS, so as to obtain AuNRs-Apt or AuNRs-RC. The AuNRs-Apt or AuNRs-RC was stored at 4° C. for later use.
(3) The AuNRs-Apt and AuNRs-RC prepared in step (2) were mixed in an equal ratio of 1:1, oscillated and incubated for 12 h at the room temperature, so as to prepare the gold nanorod dimer.
(4) A final concentration of 10 μM of Raman signal molecule 4-MBA was added into a gold nanorod dimer solution prepared in step (3), oscillated and incubated for 4 h at the room temperature, so as to obtain a Raman enhanced substrate solution. The Raman enhanced substrate solution was stored at 4° C., and was configured to subsequently detecting wheat gluten.

### EXAMPLE 2

### Standard Curve of Detection of Wheat Gluten

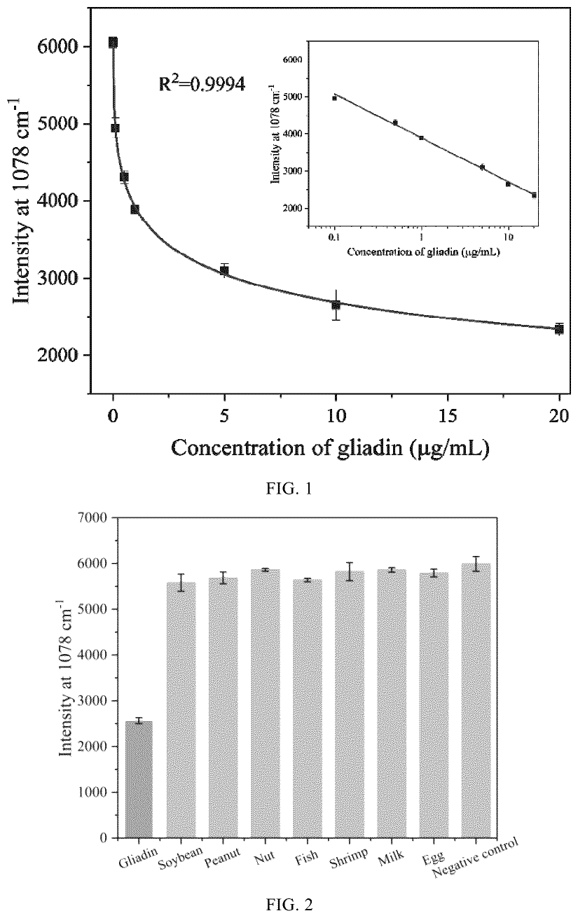

Detection performance of a kit was explored with wheat gliadin as a representative. Detection steps were as follows: 20 μL of reagent I (Raman enhanced substrate solution obtained in Example 1) was added into 1 μL of sample solution, and incubated for 45 min at a room temperature. 10 μL of solution was dropwise added onto a quartz sheet, and the quartz sheet was dried. A Raman signal intensity was measured with a Raman spectrometer. A laser wavelength for Raman intensity measurement was 785 nm. A standard curve (as shown in FIG. 1) was drawn according to the Raman signal intensity of the Raman signal molecule at 1078 cm⁻¹, and a total content of the wheat gliadin in a sample was calculated.

A kit has stable detection, short time consumption, a detection limit that may be as low as 35.06 ng/mL, and a quantitation limit of 116.87 ng/mL. Detection results may be quantitatively detected by means of a portable Raman spectrometer. All experiments were repeated three times. Results had excellent reproducibility.

### EXAMPLE 3

### Stability and Accuracy of Kit to Detect Standard Sample

Three sets of different concentrations of wheat gliadin standard samples were prepared separately. Each set of samples were detected with a kit. Three parallel samples were set for each set of samples, and experiments were repeated three times. An inter-batch difference and an intra-batch difference were calculated. Results showed that the inter-batch difference was 2.27%-8.50%, and the intra-batch difference was 3.01% -6.58%. The detection method had excellent stability and accuracy. Specific results were shown in Table 1 below:

TABLE 1

| | Standard sample | Detection result | Relative standard |
|---|---|---|---|
| | amount (μg/mL) | (μg/mL) | deviation (RSD) (%) |
| Intra-batch | 0.5 | 0.48 ± 0.03 | 6.58 |
| | 1 | 0.98 ± 0.03 | 3.01 |
| | 5 | 5.04 ± 0.20 | 3.95 |
| Inter-batch | 0.5 | 0.52 ± 0.04 | 8.50 |
| | 1 | 0.97 ± 0.03 | 3.24 |
| | 5 | 4.99 ± 0.11 | 2.27 |

### EXAMPLE 4

### Specificity of Standard Sample Detected by Kit

A soybean protein extract, a nut protein extract, and a peanut protein extract were extracted by using a Kaiji plant whole protein extraction kit, a fish protein extract and a shrimp protein extract were extracted by using a Kaiji plant whole protein extraction kit, and 10 mg/mL of solution was prepared by milk powder and whole egg powder, to obtain a protein extract. According to the steps in Example 2, the specificity of the kit was verified. Results were shown in FIG. 2. A surface enhanced Raman scattering (SERS) intensity of blank samples (peanut, soybeans, nut, fish, shrimp, milk, and egg) was close to negative control, which indicated that the kit had excellent specificity for detecting wheat gluten.

### EXAMPLE 5

### Accuracy of Kit to Detect Standard Sample

0.5 mL of a whole protein extract of soybean, peanut and nut extracted in Example 4 was added into different amounts of gliadin separately, to make final concentrations of the gliadin to be 0.5 μg/mL, 1.0 μg/mL and 5.0 μg/mL, and three parallel samples were set for each concentration, to determine a recovery rate of a blank standard sample. Detection results were shown in Table 2. In a concentration addition range of 0.5 μg/mL-5.0 μg/mL, the recovery rate was 89.64%-108.49%. The detection method had excellent accuracy. Specific results were shown in the table below:

TABLE 2

| | Standard sample | Detection result | |
|---|---|---|---|
| Sample | amount (μg/mL) | (μg/mL) | Rate of recovery |
| Soybean | 0.5 | 0.51 ± 0.02 | 102.81% |
| | 1.0 | 0.91 ± 0.10 | 90.62% |
| | 5.0 | 4.81 ± 0.02 | 96.17% |
| Peanut | 0.5 | 0.48 ± 0.01 | 95.71% |
| | 1.0 | 1.08 ± 0.03 | 108.49% |
| | 5.0 | 5.12 ± 0.34 | 102.34% |
| Nut | 0.5 | 0.45 ± 0.04 | 89.64% |
| | 1.0 | 0.98 ± 0.03 | 98.13% |
| | 5.0 | 4.73 ± 0.10 | 94.53% |

In the description of the description, the description with reference to terms such as “an example”, “some examples”, “instances”, “particular instances”, or “some instances” means that specific features, structures, materials, or characteristics described in combination with the examples or instances are encompassed in at least one example or instance of the present disclosure. In the description, the schematic expressions of the terms described above should not be understood as necessarily referring to the same example or instance. Moreover, the specific features, structures, materials or characteristics described can be combined in a suitable manner in any one or more examples or instances. In addition, those skilled in the art can bond and combine different examples or instances described in the description.

Although the examples of the present disclosure have been shown and described above, it can be understood that the above examples are illustrative and cannot be understood as limitations to the present disclosure. Those of ordinary skill in the art can make changes, modifications, substitutions, and variations to the above examples within the scope of the present disclosure.

What is claimed is:

1. A kit for detecting a content of a wheat gluten in a food, comprising a Raman enhanced substrate solution and a quartz sheet, wherein the Raman enhanced substrate solution comprises a gold nanorod dimer and a Raman signal molecule, wherein the gold nanorod dimer is formed by self-assembling a gold nanorod functionalized by an aptamer and a gold nanorod functionalized by an aptamer complementary chain.

2. The kit of claim 1, wherein the aptamer has the nucleic acid sequence as shown in SEQ ID NO: 1, and the aptamer complementary chain has the nucleic acid sequence as shown in SEQ ID NO: 2.

3. The kit of claim 1, wherein a molar ratio of the gold nanorod to the aptamer or the aptamer complementary chain is 1:60.

4. The kit of claim 1, wherein a length-diameter ratio of the gold nanorod is 3.7.

5. A method for detecting a content of a wheat gluten in a food by using the kit of claim 1, comprising:
adding 20 μL of the Raman enhanced substrate solution into 1 μL of a sample solution to be detected to obtain a Raman enhanced substrate-containing sample solution, carrying out an incubation on the Raman enhanced substrate-containing sample solution at a room temperature to form a mixed solution, dropwise adding 10 μL of the mixed solution on the quartz sheet to obtain a resulting quartz sheet, and drying the resulting quartz sheet to obtain a dried quartz sheet; and
placing the dried quartz sheet into a Raman spectrometer for measuring a Raman signal intensity and calculating a total content of the wheat gluten in a sample to be detected.

6. The method of claim 5, wherein an incubation time is 45 min.

7. The method of claim 5, wherein a laser wavelength for measuring the Raman signal intensity is 785 nm.

8. The method of claim 5, wherein the Raman signal intensity is a signal intensity of the Raman signal molecule at 1078 cm⁻¹.

9. The method of claim 5, wherein in the kit, the aptamer has the nucleic acid sequence as shown in SEQ ID NO: 1, and the aptamer complementary chain has the nucleic acid sequence as shown in SEQ ID NO: 2.

10. The method of claim 5, wherein in the kit, a molar ratio of the gold nanorod to the aptamer or the aptamer complementary chain is 1:60.

11. The method of claim 5, wherein in the kit, a length-diameter ratio of the gold nanorod is 3.7.


SEQUENCE LISTING (2 sequences)

SEQ ID NO: 1 - DNA, 60 bp, synthetic construct
  source 1..60: mol_type = other DNA
ccagtctcccgtttaccgcgcctacacatgtctgaatgccttttttttttttttttttsh

SEQ ID NO: 2 - DNA, 50 bp, synthetic construct
  source 1..50: mol_type = other DNA
shttttttttttttttttttggcattcagacatgtgtaggcgcggtaaac